(19) United States
(12) Patent Application Publication
(10) Pub. No.: US 2008/0146674 A1
(43) Pub. Date: Jun. 19, 2008

(54) USE OF GENTIAN VIOLET IN TREATMENT OF ATOPIC DERMATITIS

(76) Inventors: E. William Rosenberg, Memphis, TN (US); Robert B. Skinner, Memphis, TN (US)

Correspondence Address: PEARNE & GORDON LLP, 1801 EAST 9TH STREET, SUITE 1200, CLEVELAND, OH, 44114-3108, US

(21) Appl. No.: 11/956,054

(22) Filed: Dec. 13, 2007

Related U.S. Application Data
(60) Provisional application No. 60/875,095, filed on Dec. 15, 2006.

(51) Int. Cl. A61K 31/13; A61P 17/00

(52) U.S. Cl. 514/641

(57) ABSTRACT

A composition and method for treating a skin problem, particularly atopic dermatitis, the composition comprising an antiseptic dye, preferably gentian violet, in a vehicle effective for topical administration.

### CROSS-REFERENCE TO RELATED APPLICATIONS

This application claims the benefit of U.S. Provisional Patent Application Ser. No. 60/875,095 filed Dec. 15, 2006, the contents of which are incorporated herein by reference in their entirety.

### FIELD OF THE INVENTION

The present invention relates generally to topical compositions comprising antiseptic dyes for treating skin problems, and, more particularly, to compositions comprising up to about 0.05 weight percent of an antiseptic dye such as gentian violet to treat skin problems.

### DESCRIPTION OF THE RELATED ART

Atopic dermatitis (also known as atopic eczema or eczema) is a common and important skin problem in infants and young adults. It occurs most frequently in families and individuals who suffer, also, from asthma and/or hay fever. All of those conditions are thought to represent an inherited tendency for an abnormal or excessive immune reactivity to various environmental antigens. Microbial products associated with Staphylococcus aureus (S. aureus) colonization of skin are now thought to be an important provocative factor in atopic dermatitis. This is not the same as saying that the itchy, scratched skin of atopic dermatitis is prone to become secondarily infected with S. aureus (which can happen). Rather, it is thought that smaller numbers of S. aureus colonize the skin of atopic dermatitis individuals and evoke an abnormal response.

In the prior art it has been suggested to use 0.3% aqueous gentian violet as a topical treatment for atopic dermatitis. See Brockow, K., et al., “Effect of Gentian Violet, Corticosteroid and Tar Preparations in Staphylococcus-aureus-Colonized Atopic Eczema,” Dermatology, Vol. 199, pgs. 231-236 (1999), the entire contents of which are incorporated herein by reference. However, gentian violet is a blue dye that, at 0.3% concentration, stains the skin and tends to stain anything else (i.e. household items, clothing, furniture) that it comes into contact with when the patient who has had it topically applied goes about his or her normal daily activities. In other words, topically applied 0.3% gentian violet is very messy, which has strongly discouraged its use. There is a need for a more effective treatment.

### SUMMARY OF THE INVENTION

In accordance with one aspect of the invention, there is provided a composition for topical application comprising an antiseptic dye being present in the range of about 0.005 to about 0.05 weight percent of the composition. In a further aspect, the antiseptic dye is gentian violet, carbol fuschin dye, Castellani's paint and/or mixtures thereof. In still another aspect of the invention, the composition may further comprise an effective concentration of a topical steroid, corticosteroid, calcineurin inhibitor, skin barrier repair agent, topical lubricant, emollient, moisturizer and/or mixtures thereof.

In another aspect of the invention, there is provided a method of treating a skin problem, such as for example atopic dermatitis, comprising applying a composition comprising an antiseptic dye in the range of about 0.005 to about 0.05 weight percent of the composition.

### DETAILED DESCRIPTION OF THE PREFERRED EMBODIMENTS OF THE INVENTION

When a range, such as 5-25, is given, this means preferably at least 5 and, separately and independently, preferably not more than 25. Percent or % means weight percent unless otherwise indicated or apparent or as known in the art.

The compositions of the present invention are useful in treating skin problems, such as varying types of dermatitis such as atopic dermatitis, any skin irritation caused by S. aureus, rashes, psoriasis, insect stings, cuts, aging skin, dry skin, eczema, itchy skin, red skin, inflamed skin, and cracked skin.

The present invention is directed to compositions for topical application comprising antiseptic dyes. The inventive compositions comprise antiseptic dyes at a level to be effective for treating skin problems; suppressing colonization of bacteria, such as S. aureus, or the lessening of the likelihood of a patient spreading bacteria, such as S. aureus, and avoiding staining from the dyes, such as on a body surface (e.g., skin) of a human. Examples of antiseptic dyes are gentian violet, carbol fuschin, Castellani's paint and the like.

The composition of the present invention may be applied in any form, such as a solid, aqueous solution or aerosol. Any suitable carrier or vehicle effective for topical administration to a patient as know in the art may be used, such as, for example, a cream base, creams, liniments, gels, lotions, ointments, foams, solutions, suspensions, emulsions, pastes, aqueous mixtures, sprays, aerosolized mixtures, oils such as Crisco®, soft-soap, as well as any other preparation that is pharmaceutically suitable for topical administration on human and/or animal body surfaces such as skin or mucous membranes. In addition, vehicles suitable for topical application may be in the form of a solid, such as in an adhesive-backed patch, sticks, bar-soap or moisturizer bar, as a cosmetic or as a powder. As part of the carrier or vehicle, other customary additives, such as preservatives, can be included in conventional amounts. The ingredients of the composition or formulation can be blended and combined in a conventional manner to provide the inventive composition as described herein.

It has been discovered that compositions comprising an antiseptic dye such as gentian violet can be effective in treating and reducing the signs and symptoms of skin problems such as atopic dermatitis in very low concentrations. The composition can comprise an antiseptic dye such as gentian violet being present in the range of about 0.005 to about 0.05, preferably about 0.01 to about 0.05, preferably about 0.02 to about 0.05, preferably about 0.03 to about 0.05, or about 0.04 to about 0.05 weight percent, as measured relative to the total of the composition. The composition preferably comprises a carrier as mentioned above. The low concentration of an antiseptic dye such as gentian violet avoids the messiness and staining problems of a higher concentration; thus a low concentration antiseptic dye composition can avoid the tendency of the higher concentration products to stain both skin and clothing. Other known antiseptic dyes, such as old time antiseptic dyes, can be substituted for gentian violet as discussed above. For example, carbol fuschin dye, Castellani's paint, or the like can be employed in the composition, preferably in the same concentration or weight percent as gentian violet. Preferably the gentian violet and/or other antiseptic dye is provided in a concentration which is effectively non-staining and substantially non-staining. Both a 0.3% concentration of gentian violet and a 0.2% concentration of gentian violet are not effectively non-staining. The above compositions comprising a concentration or weight percent of gentian violet of about 0.05% and less are effectively non-staining.

The unexpected and surprising usefulness of the invented compositions comprising a low concentration of an antiseptic dye such as gentian violet for treating skin problems represents a distinct advantage as a skin therapeutic agent: it is readily prepared, has a long record of safety (1% solutions are painted in infants' mouths for the treatment of thrush), is stable, and, unlike topical antibiotics, does not lose its effectiveness because of the emergence of drug resistant bacterial strains.

In one embodiment, an antiseptic dye such as gentian violet, in the concentrations or weight percents described above, can be combined in a vehicle or carrier with one or more other agents (in their conventional concentrations or weight percents) conventionally or currently used in the treatment of skin problems, such as atopic dermatitis, including but not limited to, topical steroids, corticosteroids and hydrocortisone (typical concentrations being 0.01-4%, more preferably 0.05-2.5%); calcineurin inhibitors such as tacrolimus (Protopic®, such a 0.1%) and picrolimus (Elidel®, such as 1%); skin barrier repair agents, such a Mimyx® and Atopiclair™; topical lubricants, such as cold cream, Aquaphor® and Eucerin®; emollients, moisturizers, antibiotics, antihistamines, anti-inflammatory drugs, anti-viral drugs, anti-fungal drugs and growth factors. Such a combination composition might include, for example, a composition comprising 0.02 wt. % gentian violet and 2 wt. % hydrocortisone in a carrier such as Crisco®. Other suitable carriers could be, for example, Mimyx® cream, or Elidel®.

In another embodiment of the present invention, an antiseptic dye such as gentian violet and/or other old time antiseptic dye can be combined in a concentration of about 0.01 to 0.0005 weight percent or less with a liquid soap or bar soap. Atopic dermatitis patients can wash their whole body with the soap every day and rinse it off as per normal bathing. The result is that their whole body is treated with the low concentration treatment every day; this will suppress S. aureus on their skin and lessen the likelihood of spreading S. aureus to other family members or others in close contact with the patient. The concentration of the antiseptic dye is lower since (a) it treats the whole body, not just the affected areas, and (b) the treatment is every day for long periods of time or continuously. The other conventional agents mentioned above can optionally be added to the liquid or bar soap.

The inventive compositions described herein can be applied in a conventional manner to a human or animal body surface, such as skin or mucous membranes. It can be rubbed, sprayed, such as in an aerosol, painted, adhered or applied in any other conventional manner onto a body surface. Preferably, a composition is applied to a body surface (e.g., skin) according to a periodic regime (e.g., daily, weekly, monthly) for a period of time sufficient to treat a skin problem. For example, a composition can be applied about or at least once daily or less preferably at some other suitable interval.

Various aspects of the invention are illustrated in the following Examples.

### EXAMPLE 1

10 year-old male patient with eczema since birth. He was treated with allergy shots for 3½ years. Patient has hay fever and his father has eczema. Previous treatment with Elidel cream, Locoid Lipocream and Elocon cream recently without effect. His arms and legs had patchy areas of redness with scab and crust. Bacterial culture shows heavy growth of S. aureus. He was prescribed 2% hydrocortisone with 0.02% gentian violet in Crisco®. His eczema is in the best control ever, after one month of use.

### EXAMPLE 2

7½ year old male with eczema. He is currently being treated with Tobradex for his eye involvement and an Aquaphor/Glycerin/steroid mix with poor control. He has failed Elidel cream and Protopic ointment. His feet, legs and arms have thick crusted dermatitis plaques and his eyes have redness and scale. Bacterial culture grew heavy growth of methicillin-resistant S. aureus. He was prescribed 2% hydrocortisone with 0.02% gentian violet in Crisco and also Mimyx™ cream. His mother called because the Mimyx™ wasn't covered by insurance but he didn't need it because he was doing better then he ever has with the hydrocortisone/gentian violet cream. She asked why her pediatrician and allergist didn't know about S. aureus and gentian violet.

While the invention has been described with reference to preferred embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A composition for topical application, said composition comprising an antiseptic dye in a vehicle, said antiseptic dye being present in the range of about 0.01 to about 0.05 weight percent of said composition.

2. The composition of claim 1, wherein said antiseptic dye is selected from the group consisting of: gentian violet, carbol fuschin dye, Castellani's paint or mixtures thereof.

3. The composition of claim 1, said antiseptic dye present in the range of about 0.01 to about 0.03 weight percent of said composition.

4. The composition of claim 1, said antiseptic dye being gentian violet.

5. The composition of claim 1, said vehicle being oil.

6. The composition of claim 1, said vehicle being in solid form.

7. The composition of claim 6, said vehicle being a solid soap.

8. The composition of claim 1, further comprising an agent, said agent being selected from the group consisting of: topical steroid, corticosteroid, calcineurin inhibitor, skin barrier repair agent, topical lubricant, emollient, moisturizer, antibiotics, antihistamines, anti-inflammatory drugs, anti-viral drugs, anti-fungal drugs and growth factors.

9. The composition of claim 1, said composition being effective for treating a patient having a skin problem.

10. The composition of claim 9, wherein said skin problem is atopic dermatitis.

11. The composition of claim 1, said composition being effective for avoiding staining of the skin of said patient.

12. The composition of claim 1, said composition being effective for reducing skin irritation caused by bacteria.

13. The composition of claim 1, said composition being effective for reducing skin irritation caused by S. aureus.

14. A method of treating a patient having a skin problem, said method comprising administering a composition comprising an antiseptic dye being present in the range of about 0.01 to about 0.05 weight percent of said composition, said composition being in a form effective for topical administration to the skin of said patient.

15. The method of claim 14, wherein said patient is a human.

16. The method of claim 14, said antiseptic dye being gentian violet.

17. The method of claim 14, said vehicle being oil.

18. The method of claim 14, said vehicle being a solid soap.

19. The method of claim 14, said composition further comprising an agent, said agent being selected from the group consisting of: topical steroid, corticosteroid, calcineurin inhibitor, skin barrier repair agent, topical lubricant, emollient, moisturizer, antibiotics, antihistamines, anti-inflammatory drugs, anti-viral drugs, anti-fungal drugs and growth factors.

20. The method of claim 14, said skin problem being atopic dermatitis.

21. The method of claim 14, said composition further being effective to reduce the colonization of S. aureus.

22. The method of claim 14, said composition further being effective to avoid staining of said skin of said patient.

23. The method of claim 14, wherein the skin problem is selected from the group consisting of: skin irritation caused by S. aureus, rashes, psoriasis, insect stings, cuts, aging skin, dry skin, eczema, itchy skin, red skin, inflamed skin, and cracked skin.

24. The method of claim 14, said antiseptic dye being in a vehicle, wherein said vehicle is selected from the group consisting of: a cream, ointment, lotion, liniment, spray, aqueous solution, gel, foam, suspension, emulsion, paste aerosol or oil.

25. The method of claim 14, said method further comprising administering said composition to said skin of said patient according to a periodic regime for a period of time effective to said skin problem.
